(19) United States
(12) Patent Application Publication
(10) Pub. No.: US 2019/0328660 A1
(43) Pub. Date: Oct. 31, 2019

(54) NANOBUBBLES IN AN ABSORBENT MATERIAL

(71) Applicants: Zvi Yaniv, Austin, TX (US); Igor Alexander Goldman, Austin, TX (US)

(72) Inventors: Zvi Yaniv, Austin, TX (US); Igor Alexander Goldman, Austin, TX (US)

(21) Appl. No.: 16/394,430

(22) Filed: Apr. 25, 2019

Related U.S. Application Data
(60) Provisional application No. 62/662,832, filed on Apr. 26, 2018.

(51) Int. Cl. A61K 9/00; A61K 9/06; A61K 33/00

(52) U.S. Cl. CPC A61K 9/0051; A61K 33/00; A61K 9/06

(57) ABSTRACT

Gas nanobubbles are generated in a liquid and then the liquid absorbed into a liquid absorbent material, such as gas nanobubbled water into a hydrogel. The absorbent material can then later be applied to an area of biological skin for transferring of the gas nanobubbles into the tissues underlying the biological skin.

This application claims priority to U.S. provisional patent application Ser. No. 62/662,832. This application incorporates by reference U.S. provisional patent applications Ser. Nos. 62/437,920, 62/490,800, 62/662,832, and 62/551,356, and U.S. patent application Ser. No. 15/850,362.

### TECHNICAL FIELD

The present disclosure relates in general to the application of gas nanobubbles, and in particular, to therapeutic applications utilizing such gas nanobubbles in a liquid absorbed within an absorbent material.

### BACKGROUND

This section is intended to introduce various aspects of the art, which may be associated with exemplary embodiments of the present disclosure. This discussion is believed to assist in providing a framework to facilitate a better understanding of particular aspects of the present disclosure. Accordingly, it should be understood that this section should be read in this light, and not necessarily as admissions of prior art.

The human skin is a protective organ, but also one that is filled with blood vessels and nerve endings, all of which can be adversely affected by disease. For example, an inherent problem with peripheral neuropathy is the narrowing of blood vessels that supply oxygen and nutrients to the nerves in the extremities, and as a result, oxygen and nutrients are not delivered to the peripheral nerves, which eventually die. Thus, a good solution would be able to alter the situation of a patient such that the blood vessels that supply the nerves in the extremities are enlarged, allowing greater flow of blood, and additionally increasing the concentration of oxygen at these locations.

It is known that carbonated springs, which are typically hot springs with a high concentration of carbon dioxide (“CO₂”), contribute to create beautiful and healthy skin. The effect of the CO₂ is the expanding of blood vessels, and as a result naturally improving the blood flow (see, e.g., H. Hayashi et al., “Immersing Feet in Carbon Dioxide-enriched Water Prevents Expansion and Formation of Ischemic Ulcers after Surgical Revascularization in Diabetic Patients with Critical Limb Ischemia,” Ann Vasc Dis., 1(2), pp. 111-117, Oct. 24, 2008; M Hashimoto et al., “Decrease in heart rates by artificial CO₂ hot spring bathing is inhibited by β₁-adrenoceptor blockade in anesthetized rats,” J Appl Physiol, 96, pp. 226-232, Aug. 29, 2003; and M. Maeda et al., “The Effects of High Concentration Artificial CO₂ Warm Water Bathing for Arteriosclerotic Obstruction (ASO),” The Journal of The Japanese Society of Balneology, Climatology and Physical Medicine, Volume 66, Issue 3, pp. 156-164, Apr. 30, 2010, which are all hereby incorporated by reference herein). The CO₂ penetrates through the skin into the capillaries, causing the blood vessels to dilate, and frees up more oxygen that has been bound to the hemogoblin (also referred to as the Bohr Effect) (e.g., see, N. Nishimura et al., “Effects of repeated carbon dioxide-rich water bathing on core temperature, cutaneous blood flow and thermal sensation,” Eur. J. Appl. Physiol. 87, pp. 337-342, Jun. 7, 2002, which is hereby incorporated by reference herein).

Referring to the diagram of the dermis and epidermis in FIG. 1, it has also been shown that a cutaneous uptake of atmospheric oxygen contributes significantly to the oxygen supply of the human dermis and epidermis (see, e.g., M Stucker et al., “The cutaneous uptake of atmospheric oxygen contributes significantly to the oxygen supply of human dermis and epidermis,” Journal of Physiology (2002), 538.3, pp. 985-994, Feb. 1, 2002, which is hereby incorporated by reference herein). In a study, it was shown that under normal conditions, atmospheric oxygen can supply the upper skin layers at a depth of 0.25-0.4 millimeters. As a result, if one can supply oxygen that is presented to the skin in the form of nanobubbles (e.g., on the order of tens of a nanometer in diameter (e.g., ultra-fine nanobubbles (“UFNB”) less than 1 micron), which is compatible with the typical dimensions of skin pores (which have an average diameter of 50 μm)), the oxygen can be directly permeable to the nerves in the dermis (e.g., see, M Stucker et al. previously referenced and D. Ladizinsky et al., “New insights into oxygen therapy for wound healing,” Wounds, 22(12), pp. 294-300, December, 2010, which are hereby incorporated by reference herein). Furthermore, oleic acids incorporated in a material, which can be topically applied, are known to increase the diameter of skin pores.

In U.S. patent application Ser. No. 15/850,362, there is disclosed the utilization of gas nanobubbles coming in contact with human skin for therapeutic reasons. Further described is how nanobubbles can come in contact with skin using a nanobubbler to produce or generate gas nanobubbles (e.g., oxygen, CO₂, etc.), in a liquid (e.g., water) and then the liquid containing the gas nanobubbles is put in contact with the skin.

Though this approach can be utilized in many applications, such as to treat peripheral neuropathy, wound irrigation, hypoxia, etc., there may be applications that require a different way to bring gas nanobubbles in contact with skin.

### BRIEF DESCRIPTION OF THE DRAWINGS

FIG. 1 illustrates a diagram of the dermis and epidermis of biological skin.

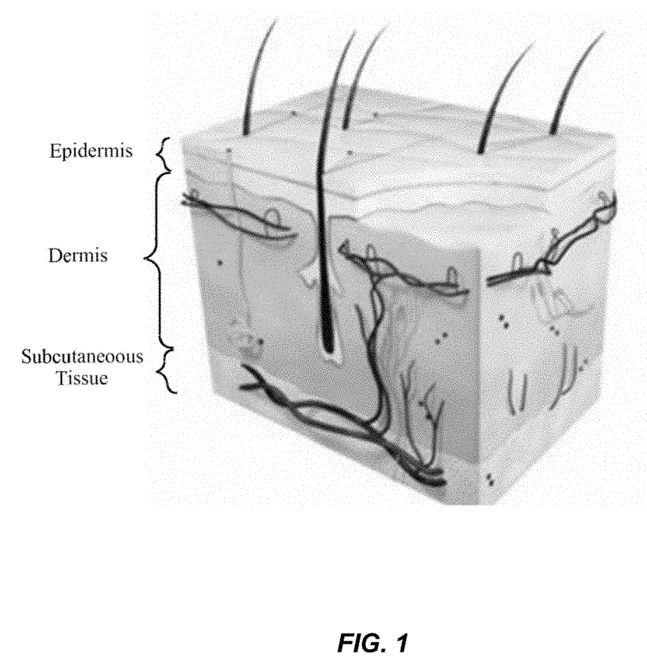

FIG. 2 shows a graph of bubble density and diameter.

FIG. 3 illustrates a schematic pertaining to the charging of gas nanobubbles.

FIG. 4 illustrates an apparatus configured in accordance with embodiments of the present disclosure.

FIGS. 5-7 illustrate an apparatus configured in accordance with embodiments of the present disclosure.

FIG. 8 illustrates of flowchart diagram configured in accordance with embodiments of the present disclosure.

FIG. 9 schematically illustrates an exemplary process for making a hydrogel that has absorbed a liquid containing gas nanobubbles in accordance with embodiments of the present disclosure.

FIGS. 10A-10B illustrate exemplary applications utilizing an absorbent material that has absorbed or been infused with a liquid containing gas nanobubbles.

FIG. 10C schematically illustrates an exemplary process demonstrating coating of a gas nanobubbled absorbent material onto a substrate.

### DETAILED DESCRIPTION

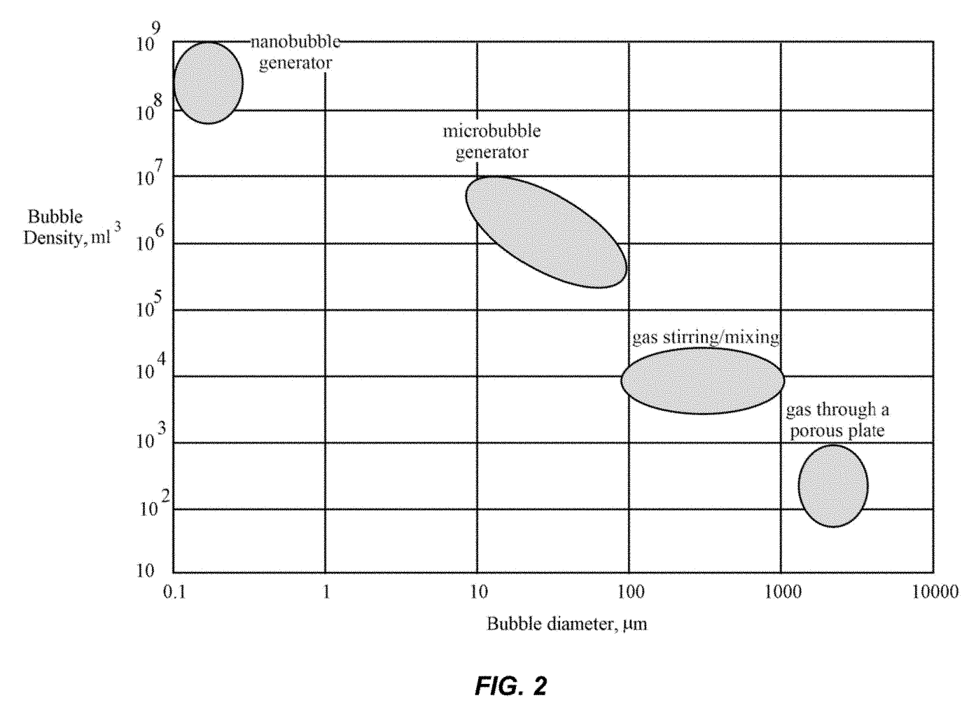

Referring to FIG. 2, nanobubbles generated in accordance with embodiments of the present disclosure are gas-filled bubbles within a liquid having a diameter less than 100 nm. Some define nanobubbles as bubbles in a liquid with a diameter smaller than 1 μm and larger than 1 nm. Other embodiments of the present disclosure may utilize nanobubbles generated, absorbed, or infused in materials, such as gels and lotions, having viscosities less than or equal to 100,000 cP. Furthermore, creams with viscosities greater than 100,000 cP or less than 600,000 cP can also be absorbed/injected/infused with nanobubbles for use in embodiments of the present disclosure. Additionally, ointments having viscosities greater than or equal to 600,000 cP can be absorbed/injected/infused with nanobubbles by increasing their temperature in order to lower their viscosity.

Each bubble is surrounded by an interface with different properties than the bulk liquid. Bubbles with a diameter less than 1 micron may be referred to herein as “ultra-fine bubbles” (“UFB”), which include the family of nanobubbles as defined above.

The surface area of a bubble is inversely proportional to the bubble diameter. As an example, for 100 nm diameter bubbles, a 1 ml liquid can contain about 2×10¹⁵ such bubbles, with a surface area that is 1,000 times (240 m²) more than the surface area of 1 mm diameter bubbles in the same volume of liquid (e.g., 2×10⁶ bubbles with a total surface area of 0.24 m²). “Fine bubbles” have inflexible surfaces (for example, as in high pressure balloons) that limit distortions, while larger bubbles have flexible surfaces (for example, as in low pressure balloons) and can break up relatively easily.

The buoyancy of larger bubbles causes them to rise to the surface of the liquid. Although the behavior of bubbles in liquids is complex, the rising rate of bubbles can be approximated using Stokes's equation:

R=μgd²/18μ

where R represents the rise rate, p represents the density, g represents gravity, d represents the bubble diameter, and μ represents the dynamic viscosity (Pa×s). Accordingly, a 25 μm diameter bubble will rise at a rate of ˜2.3 cm/min, while nanobubbles will rise much more slowly than their Brownian motion. Since nanobubbles are dominated by Brownian motion rather than behaving according to Stoke's equation, they diffuse into the liquid with minimal or no buoyancy effects.

The generation of nanobubbles can be accomplished by vigorously mixing a combination of gas and a liquid, which will generally produce bubbles with a wide range of diameters. Referring to FIG. 2, smaller bubbles when generated have a higher concentration than larger bubbles. Nanobubbles are generated at the highest density (e.g., see, M Chaplin, “Water Structure and Science,” downloaded from the Internet from URL: http://www1.1sbu.ac.uk/water/nanobubble.html, (last updated on Nov. 9, 2017), which is hereby incorporated by reference herein).

The degree of saturation next to a bubble depends on the gas pressure within the bubble. The energy cost of bubble formation depends on the interfacial area and is governed by the bubble's surface tension. Smaller bubbles have higher internal pressure and release gas to dissolve under pressure into an under-saturated liquid. Larger bubbles grow by taking up gas from a super-saturated liquid. As a result, smaller bubbles shrink and larger bubbles grow. As bubbles rise, the pressure on them drops, and consequently they enlarge and rise faster. Nanobubbles rise much slower due to low buoyancy and their random Brownian motion. The overall behavior of nanobubbles is complex due to the shrinking effects that are in competition to the effect of buoyancy. Consequently nanobubbles have a tendency to diffuse into the liquid; they do not rise and they never accumulate in the upper part of the liquid.

Additionally, the electrostatic interaction between nanobubbles can be large enough to avoid coalescence. Electrostatic interaction will slow any rise even more. The zeta potential is generally negative and mostly independent of the bubble diameter. The zeta potential depends strongly on the pH and the dissolved salt concentrations whereby increased ionic strength reduces zeta potential. As all the bubbles are similarly charged, their coalescence is discouraged. The zeta potential of a bubble can be determined from its horizontal velocity in a horizontal electric field:

v=ζϵ/μ

where ζ represents the zeta potential, ϵ represents the permittivity, and μ represents the dynamic viscosity.

According to the Laplace equation, the following is the pressure inside a gas bubble for soluble gases:

Pᵢₙ=Pₒᵤₜ+4γ/d

where Pᵢₙ represents internal pressure, Pₒᵤₜ represents external pressure, γ represents surface tension, and d represents bubble diameter. The expression 4γ/d is defined as the excess pressure.

The controversy over nanobubbles' existence is based on the fact that as the diameter of the bubble is in the range of nanometers, the internal pressure will be very high, significantly reducing their lifespan. By reducing the surface tension utilizing a surface-active agent (e.g., a surfactant), which coats the surfaces of the nanobubbles, the excess pressure can be lowered in order to stabilize the bubbles, which has the effect of lengthening the lifetime of the nanobubbles in the liquid. The concentration of surface-active agents (e.g., surfactant) may also be used to stabilize/regulate the bubble size (e.g., see M Chaplin, previously cited herein), such as limiting the growth or shrinkage of the nanobubbles.

However, the Laplace equation may not hold at small diameters such as nanobubbles. For nanobubbles, the calculated internal gas pressure should cause an almost instantaneously dissolution, but as nanobubbles are now known to exist for long periods, the existing basic theories may be insufficient.

Although nanobubbles are smaller than the wavelength of light and therefore too small to be visible to the naked eye or standard microscope, they can be visualized by backscattering of the light from a laser. Nanobubbles can be also observed by dynamic light scattering (“DLS”). The fluctuation of the scattering of laser light travelling through the sample liquid is due to the Brownian motion whereby larger bubbles are showing greater scattering but lower fluctuations. Analysis of the total signal gives both the concentration and size distribution of the nanobubbles. Other detection methods include cryoelectron microscopy (“cryo-EM”) and resonant mass measurement.

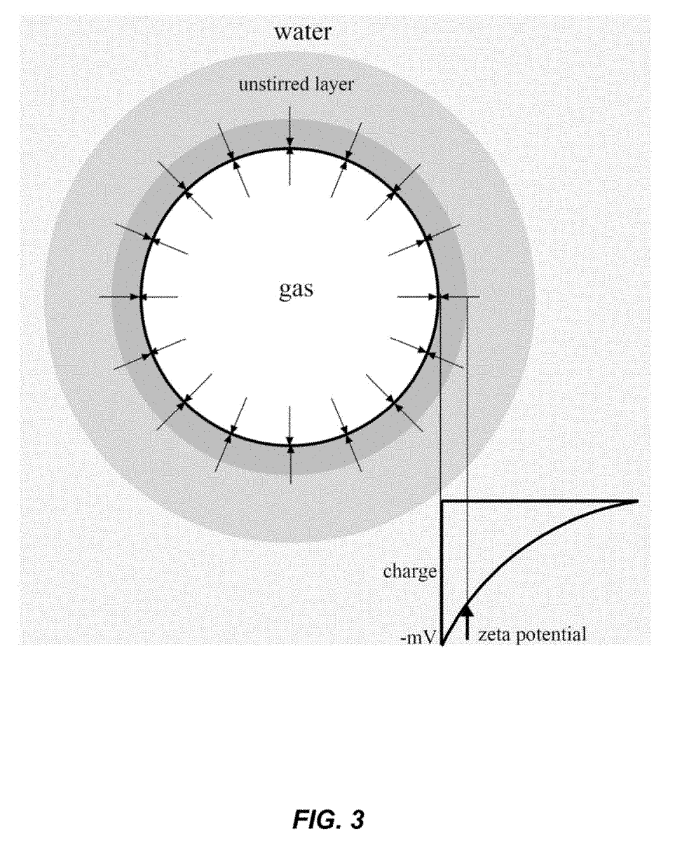

Referring to FIG. 3, a likely reason for the long-lived presence of nanobubbles is that the gas/liquid interfaces of the nanobubbles are negatively charged. This charge introduces an opposing force to the surface tension, slowing or preventing dissipation of the nanobubbles. The presence of charges at the interface reduces the internal pressure and the apparent surface tension, with a charge repulsion acting in the opposite direction to the surface minimization due to the surface tension.

Surface charge can counter the surface tension preventing high pressure within the nanobubbles. It may be expected that as the nanobubble shrinks, the charge density will increase. The effect of charges at the liquid/gas interface is that the surface negative charges repelling each other are stretching out the surface of the bubble. Thus, the effect of the charges is to reduce the effect of the surface tension. The surface tension tends to reduce the surface, whereas the surface charge tends to expand it. Equilibrium will be reached when these opposing forces are equal meaning that Pᵢₙ=Pₒᵤₜ.

Pₒᵤₜ can be found to be:

Pₒᵤₜ=Φ²/2Dϵ₀)

where Φ represents the surface charge density on the inner surface of the bubble, D represents the relative dielectric constant of the gas bubble, and ϵ₀ represents the permittivity of vacuum.

The inward pressure, Pᵢₙ, due to the surface pressure is given from the Laplace equation:

Pᵢₙ=4γ/d

where γ represents the surface tension, and d represents the diameter of the bubble. Equalizing these two pressures, one can determine the charge density at different bubble diameters. For example, for nanobubble diameters of 10 nm, 20 nm, 50 nm, 100 nm, and 200 nm, the charge density is 0.14, 0.1, 0.06, 0.04, and 0.03 e⁻/nm², respectively. The surface tension reduction contributes to the stability of nanobubbles.

The charge similarity, together with the lack of van der Waals attraction, tends to prevent coalescence of the nanobubbles.

Furthermore, the nanobubbles protect each other from diffusive loss by a shielding effect, effectively producing a back pressure of gas from neighboring bubbles, which may be separated by approximately the thickness of the unstirred layer, which slows the dissolution. The slow dissolution will be even slower than expected due to higher osmotic pressure at the gas/liquid interface, which is also driving the dissolved gas near the interface back to the nanobubble.

In accordance with embodiments of the present disclosure, nanobubbles of a desired type of gas, or multiple gas types, (e.g., oxygen (O₂), nitrogen (N₂), carbon monoxide (CO), carbon dioxide (CO₂), ozone (O₃), air, nitric oxide (NO), nitrous oxide (N₂O), helium, hydrogen, nitrogen, methane, noxious gases, pure gases, mixed gases, etc., or any gas as listed at https://en.wikipedia.org/wiki/List_of_gases) are generated in a liquid, and the liquid is infused or otherwise absorbed into a matching or corresponding suitable absorbent material. Pure gases can take several different forms. They might be made up of individual atoms, such as atomic gases. For example, oxygen is a pure gas because it is made of one type of elemental molecule. Pure gases may also be compound molecules, which are composed of a bunch of different atoms. For example, carbon dioxide would be considered a pure gas, but it is also a compound molecule. Mixed gases, on the other hand, are composed of more than one kind of pure gas (e.g., NO, N₂O, etc.).

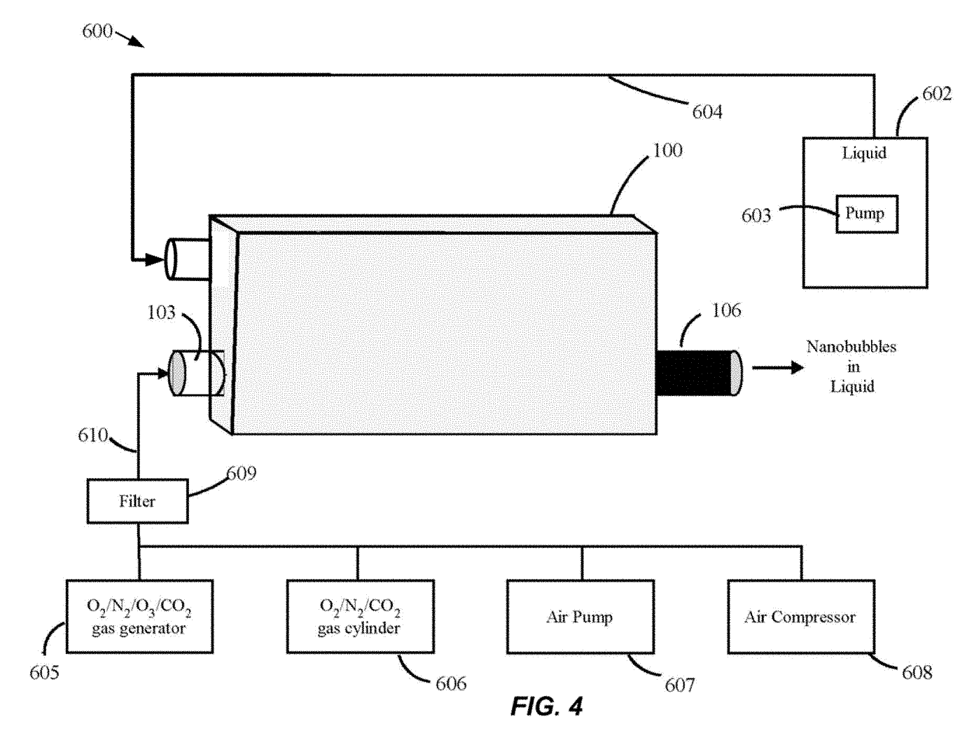

Generation of nanobubbles in a liquid may be performed utilizing any well-known nanobubble generating device, apparatus, or system (such as the nanobubbler utilizing a nozzle made of a porous carbon-based material commercially available from Anzai Kantetsu Co., LTD (e.g., see, anzaimcs.com/en/main/examplenanobubble.html, which is hereby incorporated by reference herein), which may be referred to herein as the “Anzai Device,” or as disclosed in U.S. published patent application no. 2018/0177737), or by utilizing one of the nanobubble generating systems disclosed herein with respect to FIGS. 4-7, which are further described in U.S. published patent application no. 2019/0060223, which is hereby incorporated by reference herein.

The liquid may be of any composition in which gas nanobubbles can be generated including, but not limited to, water, solutions containing water, solvents, saline, gels, foams, lotions, ointments, alcohols, oils, etc.

The absorbent material may be of any specified viscosity (including those previously listed), a hydrogel as described herein, or any material or matrix of material(s) that are absorbent of a liquid in which gas nanobubbles have been generated. Types of suitable liquid absorbent materials, include, but are not limited to, ointments, creams, sponge-type materials (e.g., cellulose sponge, etc.), all types of gels (e.g., hydrogels, silica gels, etc.), absorbent foams (e.g., Flexan, which is commercially available from Dow Hickam Pharmaceuticals, Inc.), synthetic fibers (e.g., polyester, rayon, Aquacel, which is commercially available from ConvaTech, Inc., etc.), natural fibers (e.g., cotton, wool, silk, etc.), hydrofibers that absorb water, hydrocolloids (e.g., Nu-Derm, which is commercially available from Johnson & Johnson, Inc., or RepliCare, which is commercially available from Smith & Nephew, Inc.), nano-porous materials (e.g., see Vladimir M Gun'ko et al., “Properties of Water Bound in Hydrogels,” Gels, 3, 37, Oct. 19, 2017, which is hereby incorporated by reference herein), and any other materials containing liquid absorbent molecules. For example, in the case of water, any materials having hydrophilic properties may be utilized. In addition to foams, gels, ointments, and creams, any absorbent material with a similar viscosity as these may also be utilized.

Hydrogels are gels that are composed of an aqueous dispersion medium that is gelled with a suitable hydrophilic gelling agent. Hydrogels are polar hydrophilic polymers (natural or synthetic) physically or chemically cross-linked into a three-dimensional network that can bond large amounts of water (up to 100 g per g or higher). The hydrogel hydrophilicity is due to groups such as —OH, —COOH, —COO⁻, >C═O, >CHNH₂, —CONH₂, SO₃H, etc. in the polymer network. Water plays an important role in hydrogels by supporting their integrity, solubility, and diffusion of substances, which can have importance for biomedical and biotechnological applications. For example, see R. Jayakumar et al., “Biomedical applications of chitin and chitosan based nanomaterials—A short review,” Carbohydr. Polym., 82, pp. 227-232, May 2, 2010; R. Jayakumar et al., “Novel chitin and chitosan nanofibers in biomedical applications,” Biotechnol. Adv., 28, pp. 142-150, January 2010; Y. F. Zhang et al., “O-Carboxymethyl-chitosan/organosilica hybrid nanoparticles as non-viral vectors for gene delivery,” Mater. Sci. Eng., 29, pp. 2045-2049, Apr. 8, 2009; Y.-Z. Long et al., “Recent advances in synthesis, physical properties and applications of conducting polymer nanotubes and nanofibers,” Prog. Polym. Sci., 36, pp. 1415-1442, Apr. 17, 2011; A. H. Karoyo et al., “Physicochemical properties and the gelation process of supramolecular hydrogels: A review,” Gels, 3, 1, Jan. 1, 2017, 18 pages; A. Sgambato et al., “Bioresponsive hydrogels: Chemical strategies and perspectives in tissue engineering,” Gels, 2, 28, Oct. 14, 2016, 14 pages; M Casolaro et al., “Polyelectrolyte hydrogel platforms for the delivery of antidepressant drugs,” Gels, 2, 24, Sep. 27, 2016, 15 pages; and Vladimir M Gun'ko et al., “Properties of Water Bound in Hydrogels,” Gels, 3, 37, Oct. 19, 2017, all of which are hereby incorporated by reference herein.

The water content in a hydrogel can change the hydrogel volume by swelling and shrinking, which can be purposefully influenced by external conditions. Furthermore, the water bounded in the hydrogel pores can be in a different state, strong and weak bound to the polymer network, and some of the water can be free (non-bounded).

Some hydrogels (called “smart hydrogels”) have the quality of changing their properties of absorption of water as a function of a change in an internal or external condition or parameter. This property can be utilized to accelerate and improve the release of the absorbed water including the gas nanobubbles that is in contact with the skin. For example, there are hydrogels that can change their hydrophilic properties to hydrophobic properties, as a function of a change in internal or external conditions, such as pH value (e.g., of the water), temperature, pressure, and others. This can be beneficial in applications where initially one wants the hydrogel to be hydrophilic and absorb large quantities of gas nanobubbled water, but then eventually change to hydrophobic so the water is expelled from the hydrogel, increasing the quantity of gas nanobubbled water in contact with the skin. For example, one can start by absorbing water with gas nanobubbles when the hydrogel material is very hydrophilic, but when it is desired for the gas nanobubbles to be closer to the skin, the hydrogel material can be caused to change to hydrophobic, for example by changing the pH, which will “propel” the water containing the gas nanobubbles toward the skin.

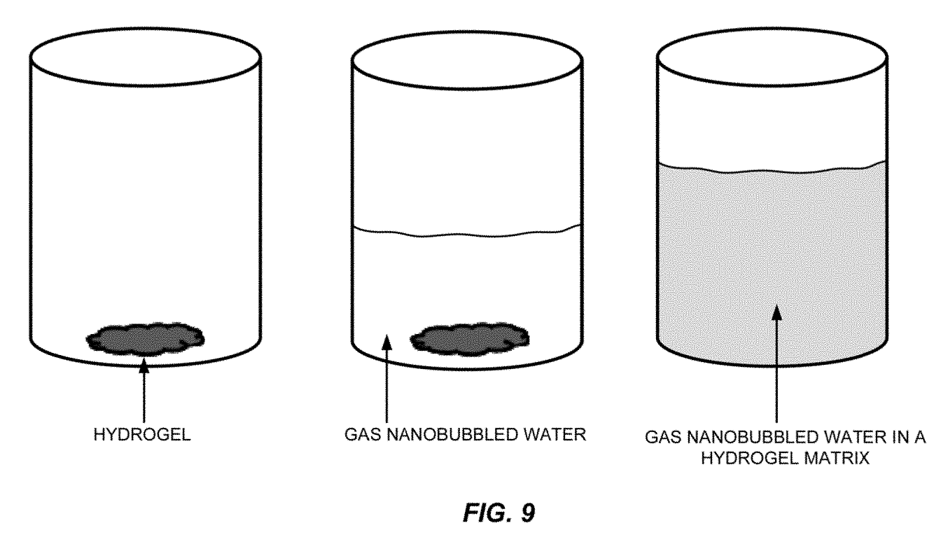

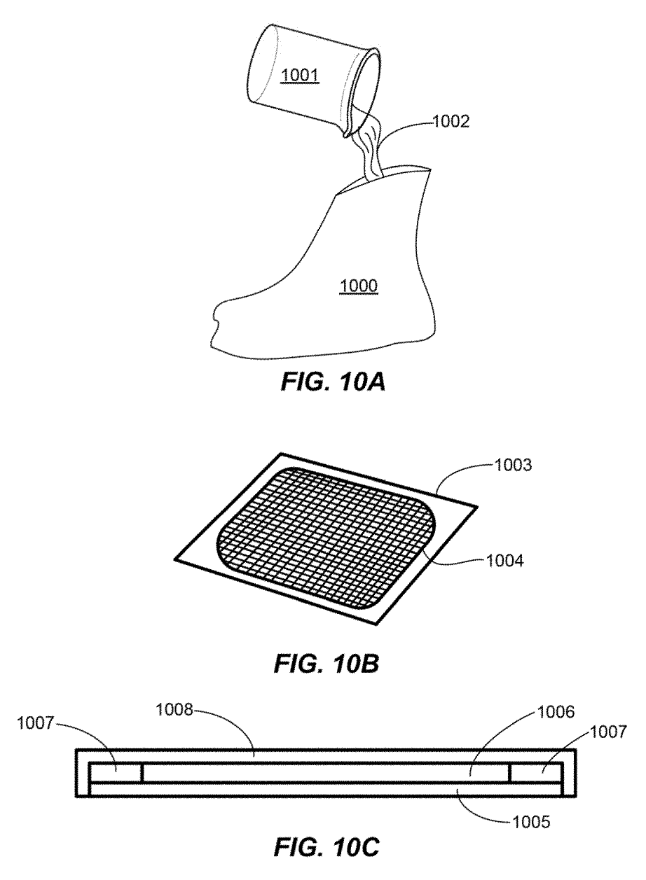

A similar principle can be utilized for storing a liquid with gas nanobubbles and then extracting the gas from the liquid absorbing material for different applications and not necessarily for bringing the material in contact with the skin. For example, see FIG. 10B and K Rehman et al., “Recent advances in gel technologies for topical and transdermal drug delivery,” Drug Dev. Ind. Pharm., 40(4), pp. 433-440, Aug. 13, 2013, which is hereby incorporated by reference herein. Drug release from hydrogels can occur from different mechanisms: diffusion and by chemical stimulation. Diffusion is regulated by movement through the polymer matrix or by bulk erosion of the hydrogel. Chemical-stimulated gels swell in response to external cues like pH and temperature or by enzymatic action and effectively open the gel pores for release of the entrapped drug (which may be contained within the gas nanobubbles). This type of mechanism can be used for targeted drug release for diseased tissues. Drug release via diffusion is more common for localized and non-specific drug release, whereas drug release by chemical stimulation may be more applicable for oral drug delivery and can offer control for selective treatment.

Additionally, embodiments of the present disclosure include other types of liquid absorbent materials that are configured to be chemically stimulated in response to external cues like pH and temperature or by enzymatic action to effectively cause the liquid absorbent material to release the gas nanobubbles from the liquid absorbent material.

There are two types of hydrogels: anionic and cationic. Due to the fact that gas nanobubbles in water (liquids) are negatively charged, a question arises about the possibility, depending on the nature of the hydrogel, that the gas nanobubbles could be pinned to the walls of the pores of the hydrogel and affect their release. In such cases, anionic hydrogels may be implemented for use because the positive pole of the water molecule will be attracted to the anions of the hydrogel structure, leaving the negative pole of the water toward the water that is free. When this occurs, the negatively charged nanobubbles will primarily remain in the free water.

As will be further disclosed herein, certain embodiments of the present disclosure supply CO₂ and/or oxygen directly to areas in the skin, and more specifically CO₂ and/or oxygen nanobubbles, which can easily penetrate into the skin pores and deep into the dermis to dilate the blood vessels that supply the peripheral nerves, while also supplying additional oxygen directly to the nerves. It is known that while oxygen has antibacterial and antimicrobial properties, ozone (O₃) is even more effective; thus, nanobubbles containing ozone can also be supplied to the skin as disclosed herein. Furthermore, embodiments of the present disclosure can also be utilized in a similar approach for wound irrigation, and even for cosmetic reasons, such as beautification of the skin.

It is important to note that when gas nanobubbles are absorbed/infused in water or in any other material, the partial pressure of the gas dissolved in the water/material will increase by a factor of between 2 and more than 10 times, and as a result, a strong force will be applied between the water/material in contact with the skin toward the inside of the skin such that the gas penetrates through the skin, and in addition will “drive” the gas nanobubbles through the pores of the skin.

FIG. 4 illustrates a block diagram of an apparatus 600 (also referred to herein as a nanobubble generator) configured to generate nanobubbles and/or infuse them into a liquid using the nanobubbler 100. The generated nanobubbles may contain any one or more desired gases (e.g., oxygen (O₂), nitrogen (N₂), CO₂, O₃, air, and/or any other desired gas, including those disclosed herein), which may be received from a source, such as from one or more of a gas generator 605 configured to supply such gas(es), one or more pressurized gas cylinders 606 (or from a cylinder that contains a compatible combination of such desired gases), an air pump 607, and/or an air compressor 608. The supplied gas may be filtered by a filter 609 before being supplied to the nanobubbler 100 at a first inlet 103. An appropriate liquid may be contained within a receptacle 602, and a pump 603 utilized to pump the liquid over the supply line 604 to a second inlet 104. The nanobubbles are generated and/or infused into the liquid. The liquid with the contained nanobubbles (also referred to herein as the “gas nanobubbled liquid”) then exits from the outlet 106, where it can then be absorbed into a liquid absorbent material (and subsequently utilized for the various applications described herein (e.g., medical, therapeutic, cosmetic, etc.)).

Note that the apparatus 600 may include a plurality of nanobubblers 100, each configured to generate nanobubbles in the liquid containing a different type of gas (e.g., oxygen (O₂), nitrogen (N₂), CO₂, O₃, ambient air, and/or any other desired gas). The liquid may be pumped through a combination (e.g., arranged in parallel or in series) of such plurality of nanobubblers 100, and then the liquid combined as it exits from each nanobubbler 401. Alternatively, a single nanobubbler 100 may be configured to generate nanobubbles in the liquid with a plurality of different gases (e.g., oxygen (O₂), nitrogen (N₂), CO₂, O₃, ambient air, and/or any other desired gas), whereby either the different plurality of gases are simultaneously passed through the diffuser 101, or in an intermittent basis, one after the other.

Exemplary Non-Limiting Nanobubbler Apparatus Specifications:
Dimensions of the diffuser 101: 160×35×22 (mm)
Pore dimensions of the diffuser 101: <1 micron
Pressure of injected gas: 5-10 psi
Liquid flow through the chamber 105: 5-15 liters/minute
Water Nozzle: Φ15 mm
Gas Nozzle: Φ6 mm
Body case material: PVC
Nanobubbler envelope dimensions: 220×50×50 (mm)

Note that such specifications for a nanobubbler may be dependent upon the particular gas, or gases, to be contained within the generated nanobubbles, and also dependent upon the viscosity of the liquid within which the nanobubbles are generated/infused.

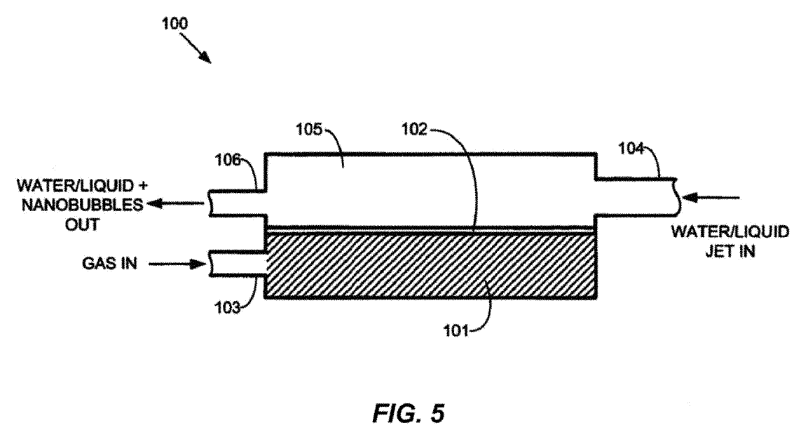

FIG. 5 illustrates an embodiment of the present disclosure implementing a nanobubbler 100 configured to have the nanobubbles emit only from one or more selected surfaces 102 such that the cross-sectional area of the liquid flow can be controlled, and as a result its pressure and flow intensity.

As shown in the illustrated cross-section of the nanobubbler 100, the gas is injected, via the inlet 103, into the porous ceramic material 101, while the liquid is injected, via the inlet 104, into the adjoining chamber 105 so that is passes across the surface 102, which is emitting the nanobubbles into the liquid. The nanobubble-infused liquid (i.e., gas nanobubbled liquid) then exits via the outlet 106.

In accordance with embodiments of the present disclosure, high quality ceramics are acquired from vendors, and then using a process described herein, the sizes of the emitting pores can be tuned as desired. In such a case, between 100 nm to 600 nm pores can be obtained that are adequate for creating nanobubbles (diameter less than 100 nm) and “minibubbles” (diameter less than or equal to 1 μm).

In accordance with certain embodiments of the present disclosure, the nanobubbler 100 may be configured with a film 102 on the top of the emitting ceramic pores that provides an external control of the diameters of the emitting pores achieving both narrower pores and a much narrower distribution of the pore diameters.

Referring to FIGS. 6-7, an exemplary film 102 that can be utilized is a UV curable epoxy (or similar UV curable polymeric materials). FIG. 7 illustrates a cross-section of the apparatus illustrated in FIG. 6 along the dashed line A-A′. In this case, the emitting surface 202 of the porous ceramic material 101 is covered (wholly or partially) by the UV curable epoxy film 102 such that before the irradiation of UV light from a UV light source 201, air (or any gas for this purpose) is continuously flowing (via an inlet 203) through the pores of the porous ceramic material 101 under high pressure such that the diameters of the respective pores created in the film 102 can be controlled (via the applied gas pressure), and then the resultant pore structure created in the film 102 can be fixed by the UV curing process, which may be performed in parallel with the gas flow. In such a way, one can achieve the necessary solution of lowering the pore diameters and also controlling the distribution (range) of pore diameters in an emitting surface of the film 102.

Other similar materials polymeric in nature may alternatively be utilized with UV curing (or any other curing method that can achieve the desired solution as explained above).

Furthermore, the apparatus 600 may be utilized to form the pores in the UV curable epoxy film.

The nanobubbler 100 may be configured to produce a combination of microbubbles and nanobubbles with a larger proportion of nanobubbles with respect to the microbubbles. The nanobubbler 100 may be made of a high-density porous ceramic (without added carbon) with pores less than 1 μm, including many around 0.5 μm. The nanobubbler 100 may have a substantially rectangular shape whereby the liquid flow through the chamber 105 is laminar and parallel to the nanobubbles, emitting surface.

In accordance with embodiments of the present disclosure, any well-known technique for manufacturing, or otherwise creating, an absorbent material in which a liquid is absorbed, or otherwise infused, into the absorbent material may be utilized. As described herein, such a liquid will contain nanobubbles generated/infused therein.

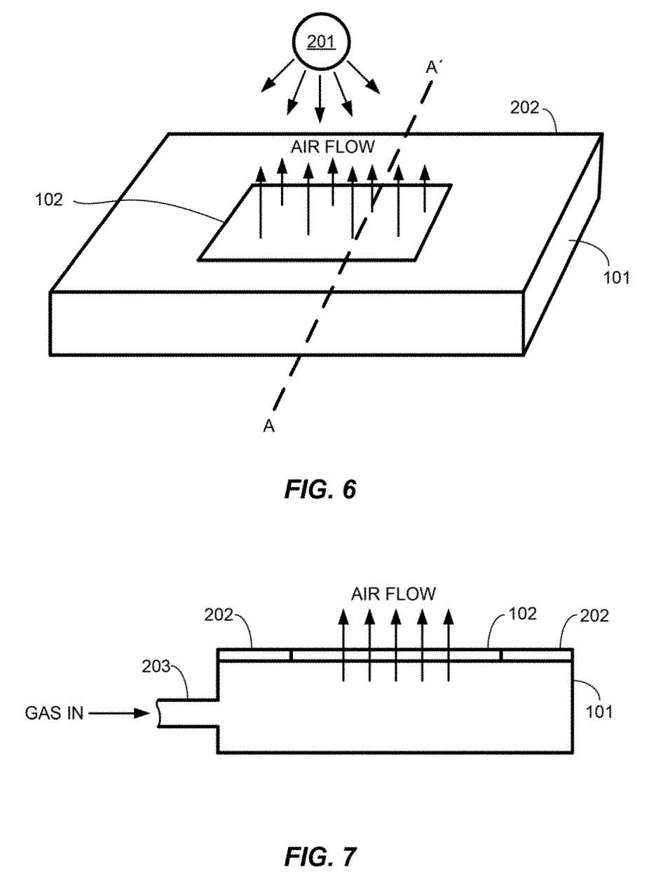

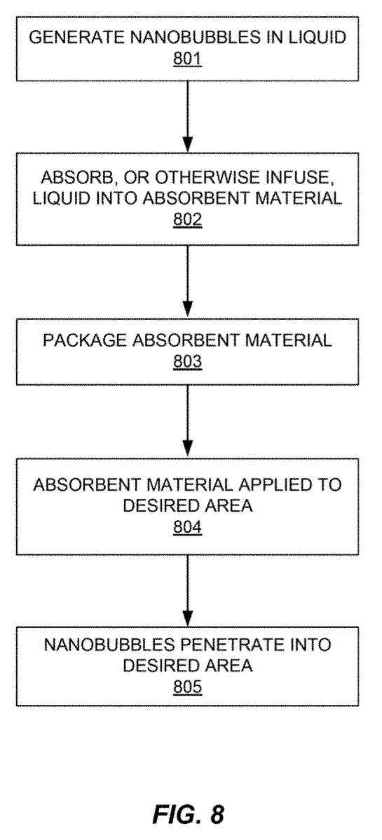

FIG. 8 illustrates a flowchart diagram of processes configured in accordance with embodiments of the present disclosure. In the process block 801, nanobubbles of one or more desired gases are generated in a liquid as described herein. In the process block 802, the liquid containing the nanobubbles is absorbed, or otherwise infused, into an absorbent material as described herein (for example, see FIG. 9).

In the process block 803, the absorbent material may then be packaged in accordance with any well-known technique for sale and/or transfer. For example, this could be water and/or gas impermeable materials in the shape of booties, socks, bags, sacks, or any suitably-shaped container, as shown in FIG. 10A. At some time in the future, in the process block 804, the absorbent material is applied to a desired area. Such a desired area may be human or other mammalian skin, but embodiments of the present disclosure are not limited to such applications. In the process block 805, the nanobubbles then penetrate into the desired area for delivery of the gas within the nanobubbles in a desired manner, such as disclosed herein.

FIG. 9 schematically illustrates a process whereby a gas nanobubbled water is absorbed into a hydrogel. In a suitable vessel or container, hydrogel and a gas nanobubbled water are brought in contact with each other. As disclosed herein, the gas nanobubbled water is absorbed into the hydrogel matrix, which may then be incorporated into a desired suitable substrate.

In accordance with certain embodiments of the present disclosure, by combining hydrogels and gas nanobubbles one can create a dressing, film, mask, or gel that contains hydrogels absorbed/infused with gas nanobubbled water (e.g., see FIGS. 10B and 10C). Due to the fact that hydrogels are an excellent water absorbent, the gas nanobubbles will also be introduced into the pores of the hydrogel, and as such will be bounded to the walls of the pores (strong bonding, weak bonding, and non-bonding). Through the process of the drying of the hydrogel and diffusion, the nanobubbles of gas can be brought in contact with the skin, open wounds, healing wounds, peripheral neuropathy skin affected areas, etc. The same can be said about any liquid-absorbent material able to absorb a liquid (other than water) containing gas nanobubbles.

Nanocomposites of hydrogel and nanobubbles can be produced (e.g., see Vladimir M Gun′ko et al., previously cited) in which changing internal or external (e.g., environmental) conditions, such as temperature, hydration, pH, ionic strength, salinity, pressure, etc., can cause such nanocomposites to exhibit a complex hierarchy of dynamic processes (e.g., hydrophilic, hydrophobic) that can be very fast and local conformational rearrangements. As a whole, the behavior of bounded and non-bounded water molecules in the micro-pores and polymers at the interfaces may depend on many factors such as topology, porosity, surface chemistry, etc., such that many structures with different water bonding qualities and configurations can be obtained.

FIG. 10A shows an exemplary application for bringing a hydrogel 1002 absorbed/infused with gas nanobubbles in contact with skin. In this non-limiting example, a flexible bootie 1000, for example made of cloth or plastic, can have a gas nanobubbled absorbed hydrogel 1002 applied inside of the bootie 1000 (e.g., poured from a container or squeezed from a tube 1001), which is then fitted over a person's foot (or the bootie fitted over the foot and then the hydrogel applied into the bootie 1000). For example, in the case of foot neuropathy, one can design a sock or booties (e.g., see FIG. 10A) that includes or is filled with a hydrogel absorbed/infused with gas nanobubbled water. Alternatively, any shaped container made of a suitable material can be utilized for holding such a hydrogel to come into contact with a person's extremities.

FIG. 10B shows another non-limiting example in which a dressing 1003, such as a transdermal patch, is packaged in a well-known manner with a gas nanobubbled hydrogel 1004. Such a dressing 1003 can be applied to a desired area of the skin.

A number of products exist for wound dressings that are infused with hydrogels. For example, the commercially available McKesson Hydrogel Impregnated Dressing is a primary dressing for wounds with light or no exudate that add or maintain moisture. The dressing helps maintain a moist wound healing environment to assist in autolytic debridement. Additionally, embodiments of the present disclosure may be incorporated with commercially available Hydrofiber technology or wound dressings.

In the case of peripheral neuropathy, special dressings can be made that are applied directly to the skin areas affected by the peripheral neuropathy and may be more comfortable and effective. Similar embodiments could be used in other applications such as cosmetics, skin treatment, gas infusion through the skin, etc.

FIG. 10C schematically illustrates a side view of a non-limiting example of another embodiment of the present disclosure demonstrating a commercial product in which an absorbent material 1006 that has absorbed or been infused with gas nanobubbles of any variety disclosed herein is applied, deposited, or coated onto a substrate 1005. An adhesive 1007 may be positioned on the substrate 1005 and around a perimeter of the absorbent material 1006. A film 1008 may enclose the absorbent material 1006 and the adhesive 1007 until the product is ready for use. The film 1008 may be made of any well-known material (aluminum foil) suitable for protecting (e.g, from light, oxygen, and bacteria) the absorbent material 1006 and maintaining its properties (e.g., moisture content). When the product is ready for use, the protective film 1008 is removed by the user. The product may then be placed against biological skin with the absorbent material 1006 contacting the skin whereby the gas nanobubbles will then be transferred into the tissues underlying the skin's surface. The absorbent material 1006 may take the form of any of the examples described herein, including, but not limited to, a hydrogel. A non-limiting example of such an application may be that the substrate 1005 is a facial mask.

Other applications related to hydrophilic materials that are very strong water absorbents (for example, hydrogels) are discussed in George Paleos, “What are Hydrogels?”, Pittsburgh Plastics Manufacturing Inc., 2012, 4 pages, and Enas M Ahmed, “Hydrogel: Preparation, characterization, and applications: A review,” Journal of Advanced Research, Volume 6, Issue 2, pp 105-121, Jul. 18, 2013, which are both incorporated by reference herein. There are many types of water absorbent materials and molecules (F. Dehghani et al., “Engineering porous scaffolds using gas-based techniques,” Current Opinion in Biotechnology, 22, pp. 661-666, May 3, 2011, which is incorporated by reference herein), and all of these materials can be produced as described herein to absorb water with nanobubbled gas or gases for many medical, agricultural, industrial, and hygienic materials.

The applications of water absorbent materials infused with nanobubbled gas or gases in water or other liquids are numerous: for example, cosmetics, hygienic products, wound care products, diapers, water-absorbent socks, and so on.

In addition to the foregoing disclosure, it should be noted that the human skin acts as a protective barrier to keep noxious substances out of the body and prevent excessive loss of water from the internal organs. Strategies have been developed to deliver drugs to the skin and surpass the skin's barrier properties. Consequently, there are many applications related to transdermal and topical drug delivery (e.g., see D. I. J Morrow et al., “Innovative Strategies for Enhancing Topical and Transdermal Drug Delivery,” The Open Drug Delivery Journal, 2007, vol. 1, pp. 36-59, which is incorporated by reference herein).

Based on the examples previously disclosed herein, cutaneous external applications of gases (such as oxygen, carbon dioxide, ozone, etc.) may have very positive effects on the health of human beings in the same way as transdermal drug delivery. A critical factor in drug delivery, for example, is the permeability through the skin (e.g., see R. J. Scheuplein, “Permeability of the Skin: A Review of Major Concepts and Some New Developments,” The Journal of Investigative Dermatology, vol. 67, issue 5, part 2, pp. 619-681, November 1976, which is incorporated by reference herein), and lately, penetration enhances were developed in particular for drug delivery systems.

In accordance with embodiments of the present disclosure, methods and devices utilizing a cutaneous external application of gases may implement skin penetration enhancer materials (also referred to herein as skin penetration agents) for the specific gases utilized such that an improved transdermal delivery can be achieved. Such skin penetration enhancers may be combined with the absorbent material that contains a gas nanobubbled liquid. For example, in the process of FIG. 8, skin penetration enhancer materials may be included with the gas nanobubbles infused into the liquid absorbent material.

As previously noted, human skin has the unique property of functioning as a physiochemical barrier; however, especially small molecules can surpass this barrier by being able to pass through the corneal layer, which is considered to form the main deterrent. In the publication J. D. Bos et al., “The 500 Dalton rule for the skin penetration of chemical compounds and drugs,” Exp. Dermatol., vol. 9, pp. 165-169, July 2000, which is incorporated by reference herein, it was asserted that the molecular weight of a compound must be under 500 Dalton to allow skin absorption. Larger molecules cannot pass the corneal layer. As a result, the most commonly used pharmacological agents applied in topical dermatological therapy drug delivery systems are under 500 Dalton (the 500 Dalton rule). For example, nanobubbles of oxygen and carbon dioxide easily fulfill this rule (oxygen having 32 Dalton and CO₂ having 44 Dalton).

As previously disclosed, gas nanobubbles can be supplied externally to the skin in a number of ways, such as when they are contained in liquids, gels, ointments, creams, etc. In accordance with certain embodiments of the present disclosure, their effectivity may be improved with implementation of a skin penetration enhancement material, be it physical or chemical (e.g., see H. Trommer et al., “Overcoming the Stratum Corneum: The Modulation of Skin Penetration,” Skin Pharmacol. Physiol., vol. 19, pp. 106-121, May 9, 2006, which is incorporated by reference herein). The main lipids in the stratum corneum are ceramides such as fatty acids and cholesterol. Gas nanobubbles supplied to the human skin, as stated above, can have an important function if, in addition to their small sizes on the order of magnitude smaller than that of the skin pores, they are embedded in formulations that also contain a skin penetration enhancer material.

The nanobubbles permeation options include sweat ducts and hair follicles, which can function as diffusion shunts with relatively easy pathways to the stratum corneum. However, other options for nanobubbles permeation are through the transepidermal route and the route via pores. The transepidermal route can be divided into the transcellular and intercellular route, whereby the more direct route is the transcellular, but the more common route for nanobubbles to penetrate the skin is the intercellular route (e.g., see J. Hadgraft, “Skin deep,” Int. J. Pharm. Investig., vol. 1, issue 1, pp. 291-299, April, 15, 2004, which is incorporated by reference herein).

There are many substances that can improve the nanobubbles gas permeation, for example dimethylsuphoxide, pyrolidones, etc. (e.g., see J. Hadgraft et al., “Transdermal Drug Delivery: Developmental Issues and Research Initiatives,” Marcel Dekker, 1989, which is incorporated by reference herein), but at least some of the properties of a penetration enhancer are that it should be non-toxic, non-irritant, and non-allergenic; it should not illicit any pharmacological activity within the body; it should be compatible and stable with the other components in the formulation, such as the vehicle for transporting the nanobubbles; it should not enhance the loss of substances from the body; the skin barrier integrity should recover rapidly; it should not be expensive; and it should be acceptable in terms of odor, color, and texture.

For example, hydration fatty acids fulfill the properties above (as long they are not used in concentrations that may irritate the skin). Fatty acids are carboxylic acids often with long unbranched aliphatic tails. Examples are lauric acid, linoleic acid, and oleic acid. Generally, unsaturated fatty acids possess a configuration that is more effective as a skin penetrator. In this configuration, a “kink” into the alkyl tail is introduced, and as a result, this configuration causes a greater disruption to the lipid layers. Furthermore, the length of the tail is also significant whereby C10 and C12 carbon chain lengths provide the greatest permeation enhancement (e.g., see B. J. Aungst et al., “Enhancement of naloxone penetration through human skin in vitro using fatty acids, fatty alcohols, and caffeine,” J. Pharmaceut, 1986; 33: pp. 255-34, which is incorporated by reference herein).

For example, oleic acid is a “kinked” fatty acid, and has a long C18 tail. It was proven by thermal analysis that the action of oleic acids disrupts the intercellular lipid packing, which was also proved using electron microscopy.

As a result, and as an example, a formulation that includes the gas nanobubbles absorbent material (e.g., a liquid, such as water, oil, saline, a film, a gel, a cream, a lotion, an aerosol, or an ointment), combined with hydration agents and oleic acids, can be a suitable formulation to optimize the nanobubble gas permeation through the stratum corneum. Other agents that can be very helpful to enhance the skin permeability are alcohols among the polyvalent alcohols; propylene glycol has co-solvent properties and may have a synergetic action in a mixture with oleic acids. These agents can be combined together, used individually, or in partial mixtures (e.g., see H. Trommer et al., previously referenced herein).

The skin can be considered to be a composite diffusion media corresponding to stratum corneum, epidermis, and a thin layer of dermis, each with its corresponding diffusion coefficient and thickness. This overlays the blood stream, which has a flow that can also affect the diffusional resistance (e.g., see R. J. Scheuplein, previously referenced herein).

Some of the gases available for creating nanobubbles in a vehicle can have a strong action against bacteria, viruses, and fungi, and can exercise significant antimicrobial activity. As previously disclosed, application of ozone nanobubbles has a therapeutic window; if the ozone nanobubbles are applied in a low concentration, they have little therapeutic effect, while higher concentrations can be toxic and irritating. In practice, the ozone nanobubbles may be applied together with oxygen nanobubbles reducing the respiratory exposure to ozone. Ozone has a relatively short lifetime, and when administered, must be produced at the point of use. In particular, ozone, due to its actions on wound pathogens, is very helpful when due to poor blood circulation and neuropathy, diabetic foot lesions result (e.g., see G. V. Sunnen, “Diabetic Wound Management: A Key Ingredient is Missing,” March 2007, 10 pages, which is incorporated by reference herein).

Within other embodiments of the present disclosure, administration of gas nanobubbles that include a contained medication (pharmacological agent can be performed (e.g., by incorporating them into capsules, a spray, ointments, gels, pills, lotions, etc.). Or, a pharmacological agent may be included in the liquid that also contains the absorbed gas nanobubbles. For example, in the process of FIG. 8, a pharmacological agent may be included with the gas nanobubbles infused into the liquid absorbent material.

For example, administration of gas nanobubbles with a contained medication in gaseous form can be performed by incorporating them with the absorbent material into capsules/pills/tablets, which are then taken by mouth through the GI system so that there is an uptake through the kidney and/or liver to be absorbed into the blood stream. Other more rapid administration of such medication contained within gas nanobubbles, for example, can be by sublingual and buccal methods. Sublingual administration involves placing a drug, a pill, a capsule, etc. under the tongue to be absorbed into the blood through the tissue there. Buccal administration involves a drug, a pill, a capsule, etc. administered between the gums and cheek where it dissolves and is absorbed into the blood stream. Both methods may come in the form of capsules, tablets, films, sprays, lozenges, etc. Gas nanobubbles can also be supplied by these methods with therapeutic effects. For example, direct delivery of oxygen, CO₂, and others into the blood stream. Other drug delivery methods can be adjusted to deliver gas nanobubbles including for example administration through the skin.

In accordance with embodiments of the present disclosure, a gas nanobubbled liquid may be produced in accordance with the process of FIG. 8 in which oxygen gas is nanobubbled into water or saline appropriate for inclusion into contact lens for eyes. Soft contacts are made of hydrophilic hydrogels. Therefore, the gas nanobubbled liquid may be incorporated into the contact lens in a similar manner as disclosed with respect to FIGS. 8 and 9.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a” and “an” mean “one or more” when used in this application, including the claims. Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D. The use of “/” between words means “and/or.”

It will be understood that particular embodiments described herein are shown by way of illustration and not as limitations of the disclosure. The principal features of this disclosure can be employed in various embodiments without departing from the scope of the disclosure.

All publications and patent applications are herein incorporated by reference to the same extent as if each individual publication or patent application was specifically and individually indicated to be incorporated by reference.

Any steps recited in any method or process claims may be executed in any order and are not limited to the order presented in the claims.

As used herein with respect to an identified property or circumstance, “substantially” refers to a degree of deviation that is sufficiently small so as to not measurably detract from the identified property or circumstance. The exact degree of deviation allowable may in some cases depend on the specific context.

The terms “or combinations thereof” and “and combinations thereof” as used herein refer to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

The use of the term “or” is used to mean “and/or” unless explicitly indicated to refer to alternatives only or the alternatives are mutually exclusive, although the disclosure supports a definition that refers to only alternatives and “and/or.”

What is claimed is:

1. A liquid absorbent material in which a liquid containing gas nanobubbles has been absorbed.

2. The liquid absorbent material as recited in claim 1, wherein the liquid is water and the liquid absorbent material is a hydrogel.

3. The liquid absorbent material as recited in claim 2, wherein the hydrogel is anionic.

4. The liquid absorbent material as recited in claim 1, wherein the liquid absorbent material is hydrophilic.

5. The liquid absorbent material as recited in claim 2, wherein the hydrogel is hydrophilic at certain internal or external conditions and hydrophobic at other certain internal or external conditions.

6. The liquid absorbent material as recited in claim 5, wherein the certain internal or external conditions are selected from the group consisting of temperature, humidity, pH, ionic strength, salinity, pressure, and a combination of any of the foregoing.

7. The liquid absorbent material as recited in claim 1, wherein the liquid absorbent material comprises molecules that absorb the liquid.

8. The liquid absorbent material as recited in claim 1, wherein the liquid absorbent material is selected from a group consisting of a foam, gel, ointment, cream, synthetic fibers, natural fibers, hydrocolloids, nano-porous materials, sponge-type materials, hydrofibers, and a combination of any of the foregoing.

9. The liquid absorbent material as recited in claim 1, wherein the gas nanobubbles are selected from a group consisting of oxygen, carbon dioxide, ozone, nitrogen, carbon monoxide, ambient air, nitric oxide, nitrous oxide, helium, hydrogen, methane, noxious gases, pure gases, mixed gases, pharmacological gases, and a combination of any of the foregoing.

10. The liquid absorbent material as recited in claim 1, further comprising a skin penetration enhancement material.

11. The liquid absorbent material as recited in claim 1, further comprising a pharmacological agent.

12. The liquid absorbent material as recited in claim 1, wherein the gas nanobubbles contain a pharmacological agent.

13. The liquid absorbent material as recited in claim 1, wherein the liquid absorbent material has a viscosity of 600,000 cP or greater.

14. The liquid absorbent material as recited in claim 1, wherein the liquid absorbent material has a viscosity of greater than 100,000 cP and less than 600,000 cP.

15. The liquid absorbent material as recited in claim 1, wherein the liquid absorbent material has a viscosity of less than or equal to 100,000 cP.

16. The liquid absorbent material as recited in claim 1, wherein the gas nanobubbles include a surface-active agent that stabilizes the gas nanobubbles within the liquid.

17. The liquid absorbent material as recited in claim 2, wherein the hydrogel is in a form of a contact lens.

18. The liquid absorbent material as recited in claim 1, wherein the liquid absorbent material is configured to be chemically stimulated in response to a change in an internal or external condition to thereby release the gas from the liquid absorbent material.

19. The liquid absorbent material as recited in claim 1, wherein the gas nanobubbles absorbed in the liquid absorbent material results in an increased partial pressure of the gas.

20. A method comprising:
generating nanobubbles in a liquid; and
absorbing the liquid into an absorbent material.
